(19) United States
(12) Patent Application Publication
(10) Pub. No.: US 2014/0228542 A1
(43) Pub. Date: Aug. 14, 2014

(54) Method For Producing Peptide Fractions And Use Thereof

(71) Applicant: Hans-Jürgen Danneel, Detmold (DE)

(72) Inventor: Hans-Jürgen Danneel, Detmold (DE)

(21) Appl. No.: 14/346,521

(22) PCT Filed: Sep. 21, 2012
(86) PCT No.: PCT/DE2012/000931

§ 371 Date: Mar. 21, 2014

(30) Foreign Application Priority Data
Sep. 22, 2011 (DE) DE102011114198.0

(51) Int. Cl. C07K 1/22; C07K 14/78; C07K 14/415

(52) U.S. Cl. CPC C07K 1/22; C07K 14/415; C07K 14/78
USPC 530/356; 530/375; 530/378

(57) ABSTRACT

The invention relates to a method for producing enriched peptide fractions from protein-containing raw materials, in which protein hydrolysates are separated using chromatography, according to the physiochemical properties thereof, by means of stationary phases with an aqueous solution as an elution agent.

The invention relates to a method for producing enriched peptide fractions from protein-containing raw materials, and use thereof.

Mixtures of peptides are created with the enzymatic hydrolysis of proteins both in the event of natural animal and microbiological digestion and in the event of the technical treatment of protein-containing raw materials with proteases or protease-containing microorganisms. The proteins in the raw materials are generally mixtures of polyamides of twenty different α-L-amino acids with chain lengths between 100 and several thousand monomer units, of which the sequence varies significantly from protein to protein. A very large number of polymer fragments are accordingly created with the proteolytic treatment of protein mixtures and even of pure individual proteins.

The peptide mixtures created with the enzymatic hydrolysis processes differ in terms of their composition according to the used raw materials, the used enzymes and the degree of hydrolysis set. The composition can be reproduced uniformly however under identical hydrolysis conditions.

For a very large number of peptides formed from protein hydrolysates, partly even in the original hydrolysates without enrichment, specific functional properties and biological activities have been demonstrated as follows:

Some peptides may have pronounced hydrophilic and hydrophobic regions and are therefore surface-active. They are suitable as surfactants or surfactant units and may have foam-stabilising, emulsifying, anti-static or cleansing properties. Some peptides may be aromatic substances per se or may be used as a basis for producing reaction aromas.

Some peptides, via their reducing or oxidising properties, can influence the degree of disulphide cross-linking of proteins.

Some peptides have antimicrobial effects.

Some peptides trigger repair mechanisms in hair and connective tissue.

Some peptides have anabolic or lipometabolism-activating, immunomodulating or blood-pressure-reducing effects.

As a result of hydrolysis of protein-containing raw materials, peptide mixtures can be produced with the aid of proteases, individually and in combination practically in unlimited variation, some of these peptide mixtures being described in the literature, some being offered commercially, for example as feedstuffs or foodstuffs, and some having properties as a result of which they can be used as functional additives in food products, cosmetic products and pharmaceutical products. An enrichment of the functional peptides or separation of undesired secondary constituents is carried out in accordance with the prior art in individual cases by precipitation methods or ultrafiltration. Both methods are relatively unspecific and allow only a separation into two fractions per method step.

Very expensive peptides for pharmaceutical applications are also fractionated chromatographically in accordance with the prior art, however the used resins are very costly, are mechanically unstable in large separating columns, and costly organic solvents are used as eluents. On the whole, these methods therefore are not considered for economical large-scale preparation of peptide mixtures, for example for food applications.

Methods for establishing, tracking and quantifying the efficacies of the peptides in the raw solutions and in the enriched fractions form part of the prior art. Aroma properties can thus be subject to sensory testing per se or after standardised reaction, and antibiotic properties can be demonstrated using the agar diffusion method, repairing properties on hair can be demonstrated using tensile strain measurements, and specific binding properties can be demonstrated by ligand chromatography. Physiological effects have been tested in animal tests or with other suitable bioassays.

Peptides in protein hydrolysates have physicochemical and physiological properties which predestine them as active components in a large number of applications in the feed, food, cosmetic and pharmaceutical field. The functional peptides in protein hydrolysates are generally only present, besides a very large excess of ineffective peptides, in such a low proportion that their effect is not particularly apparent, or, if apparent, other constituents of the mixture prevent application due to undesirable properties, such as colour or odour. With some exceptions, in which specific peptides can be isolated using very costly technology or on the basis of very specific solubility properties, there is no economical method with which protein hydrolysates can generally be separated on a large scale into fractions having sought chemical or physical properties.

Due to the findings of research performed in recent years however, a very high potential for application is predicted for functional peptides. An obstacle however for the marketing of functional peptides on a large scale lies in the fact that the functional peptides are present in the protein hydrolysates either only in very low concentrations or are accompanied by undesirable secondary components, such as colorants, bitter substances or odorous substances.

Large-scale process chromatography methods are indeed known from the field of sugar recovery and wastewater processing, in which chromatography is carried out in columns measuring more than 100 m³ for substance separations and substance purifications. These large-scale methods are only suitable however for very rough fractionation processes, since the adsorption behaviour of the used chromatography resins is not very selective and their chromatographic separation behaviour beyond these known technical applications is also unknown.

On this basis, the object of the invention is to fractionate peptide-containing mixtures economically on a large scale, to enrich peptides obtained in this way, and to separate off ineffective peptides or further undesirable components of the hydrolysates, such as colours, abnormal flavours and/or salts.

This technical problem is solved by a method for producing enriched peptide fractions from protein-containing raw materials, in which, according to claim 1, it is stipulated that protein hydrolysates are separated, according to their physicochemical properties, by chromatography via stationary phases with an aqueous solution as eluent.

It has surprisingly been found that some stationary phases of sufficient porosity used in large-scale process chromatography and in sugar technology, in wastewater processing or in chemical engineering are also able to fractionate peptides up to a molecule size of approximately 50 amino acids in accordance with the criteria basicity, acidity, hydrophobicity, molecule size and reciprocal specific interactions in order to thus obtain peptide fractions enriched in accordance with the physicochemical properties thereof.

Typical representatives of such stationary phases include divinylbenzene, cross-linked polystyrene resins with anionic or cationic functionalisations, cross-linked vinyl alcohol-styrene copolymers, or cross-linked phenol formaldehyde polycondensates, for example from the Amberlite product range from Dow Chemicals or the Lewatit product range from Lanxess.

The protein-containing raw materials and also commercially obtainable protein hydrolysates are preferably hydrolysed beforehand up to a defined degree with specific proteases, whereby peptide fragments from one to one hundred, in particular from two to twenty amino acids are further preferably obtained.

The hydrolysates can be concentrated further in the usual manner, for example by evaporation, set to the desired pH value, optionally clarified, for example by filtration, and charged with up to 0.3 bed volume (BV), preferably with 0.1 bed volume, to the chromatography columns filled with the stationary phases. Such charge solutions preferably have a concentration from 5 % to 50 % dry substance.

Elution preferably occurs with pure water without further additives, but optionally also with usually low additives of salts or solvents.

Depending on the desired fractionation, stationary phases are used that are hydroxy-functionalised, whereby separation occurs according to hydrophobicity, or that are anionically or cationically functionalised, whereby separation occurs according to ion exclusion and ion exchange.

Separation resins can be used in a versatile manner for chromatography, and the separation resins used as stationary phases for chromatography in particular are macroscopic or gel-like polymer resins which preferably have degrees of cross-linking that allow a partial penetration up to a molecule size of 10,000 Da and a gel filtration effect between 300 Da and 10,000 Da.

In some cases technical adsorbers without functionalisations can also be used advantageously as stationary phase for chromatography, for example granular carbon known from adsorber technology or pyrolysed separation resins, which may have adsorber properties similar to granular carbon due to the pyrolysis.

A binding equilibrium preferably of adsorbed peptides from the mixtures is adapted to the separation resins over the course of a number of chromatography cycles, said mixtures considerably influencing the course of the separation process by different affinities to unbound peptides.

Further process parameters include a flow rate of the chromatography between 0.2 and 4 bed volume per hour with an elution temperature between −4° C. and 98° C., in particular with an elution temperature between 70° C. and 95° C.

The sought functional property is firstly identified in the eluate of the chromatography columns when establishing a new separation. The separation resin and the separation conditions are selected in such a way that, where possible, constituents with undesired properties are not eluted in the range of the desired functionalities. The fraction ranges are determined by easily measurable physicochemical parameters of the eluate.

A chromatography process results in an enrichment of predefinable functional properties of the peptides and/or the purification of the peptides by removal of undesired secondary substances, such as colorants, bitter substances or odorous substances.

The peptide mixture thus obtained can be further fractionated by further purification steps, in particular also chromatographic purification steps, up to an arbitrary degree of purification of one or more peptides which can be used as functional substances in feedstuffs, foodstuffs, cosmetic products and as pharmaceutical active ingredients.

The non-functional fractions are combined, concentrated and fed for their original use in the food, feed, fermentation media or fertiliser field.

The essence of the invention will be explained further on the basis of practical examples, which in no way limit the invention.

### Example 1

A composition profile is created by means of an HPLC system from a commercially obtainable enzymatic wheat protein hydrolysate with approximately 90 % proportion of peptides with molecular weights between 250 and 2500 Da via an analytical separation column. The wheat protein hydrolysate is set to 50 % (w/w) aqueous solution and to a pH value of 4.5, is filtered, and for example is characterised in terms of some properties as follows:

conductivity based on % dry substance

colour component (E₄₃₀/cm) based on % dry substance

foaming behaviour of a 1% solution (foam volume after 1 min frit gassing 0.2 sample volume/s)

foam stability (half-life)

odour (sensory test)

taste (sensory test)

repairing effect on hair exposed to light, hot air and/or detergent in tensile strain test

A double-jacket gas column, ID: 35 mm, length: 1000 mm, is filled with a DVB cross-linked, macroporous, sulfonated polystyrene resin in calcium form, monodispersely and from a particle size of 0.4 mm. The double jacket is heated with the aid of a circulation thermostat to 85° C. The wheat protein hydrolysate is charged to the column at a concentration of 50% (w/w) and a volume of 50 ml, corresponding to 0.1 bed volume (BV), and chromatography is performed with hot mains water of average hardness and with a volume flow rate of 1 BV/h. After 1.5 BV elution volume, a renewed charge of 0.1 BV wheat protein hydrolysate is performed and a renewed elution with water. The process is repeated at least 10 times without regeneration of the separation resin, which is possible as often as desired in principle. The eluate of each chromatography cycle is collected in 15 fractions measuring 100 ml.

By way of example, the enrichment of the peptides with the corresponding properties can be read during the course of elution by online tracking of the pH progression, the colour gradient, the conductivity and the absorption at 214 nm.

The peptide composition profiles of the individual fractions were recorded using the above-mentioned HPLC methods.

The HPLC profiles of the individual fractions differ considerably from one another, and individual peptides are visibly enriched in different fractions.

Furthermore, the functional properties considered for characterisation of the wheat protein hydrolysate were also examined in individual fractions and compared with the starting solution.

The eluate fractions have different E₄₃₀/TS ratios, and the colour components enrich in some fractions and are removed in others accordingly. Some fractions, in contrast to the charge solution, are colourless, are not bitter and demonstrate a foam stability that is increased by 30 times with identical concentration. The hair repairing properties known and demonstrated already from the charge solution could likewise be found again in some fractions, but not in other fractions.

### Example 2

A soya protein hydrolysate with an average peptide size of 1500 Da is characterised and prepared for chromatography as described in Example 1. Chromatography is carried out by way of example in two different tests at a pH value of 5.0 and a pH value of 10.0.

A double-jacket gas column, ID: 10 mm, length: 300 mm, is filled with a DVB cross-linked, polystyrene-vinyl alcohol copolymerised separation resin without ionic functionalisations, monodispersely and from a particle size of 0.2 mm. The double jacket is cooled with the aid of a circulation thermostat to 10° C. The soya protein hydrolysate is charged to the column at a concentration of 35% (w/w) and a volume of 3 ml (0.1 BV) , and chromatography is performed with distilled water and a volume flow rate of 1 BV/h. After 1.5 BV elution volume, a renewed charge of 0.1 BV wheat protein hydrolysate is performed and a renewed elution with water.

The process is repeated three times without regeneration of the separation resin. The eluate is collected in 15 fractions measuring 3 ml. The composition profiles of the fractions of an elution cycle are recorded by way of example using the above-mentioned HPLC methods. In addition, the properties considered for characterisation of the soya protein hydrolysate are also examined in the individual fractions and compared with the starting solution.

The HPLC profiles of the individual fractions differ considerably from one another and individual peptides are enriched visibly in different fractions. In particular, a completely different fractionation is obtained in the test at pH 5.0 than in the test at pH 10.0.

The eluate fractions differ from one another by their colour components, their foaming behaviour and their sensory properties.

### Example 3

A composition profile was created by means of an HPLC system from an enzymatic collagen hydrolysate with peptide sizes <2000 Da. The collagen hydrolysate is dissolved at 40% (w/w) in water, set to a pH value of 8.0 and characterised as described in Example 1.

A double-jacket gas column, ID: 35 mm, length: 1000 mm, is filled with a DVB cross-linked, macroporous, sulfonated polystyrene resin in sodium form, monodispersely and from a particle size of 0.4 mm. The double jacket is heated with the aid of a circulation thermostat to 85° C. The collagen hydrolysate is charged to the column at a concentration of 40% (w/w) and a volume of 50 ml (0.1 BV), and chromatography is performed with hot deionised water and a volume flow rate of 1 BV/h. After 1.5 BV elution volume, a renewed charge of 0.1 BV collagen hydrolysate is performed and a renewed elution with water. The process is repeated at least 10 times without regeneration of the separation resin. The eluate is collected in 15 fractions measuring 100 ml per cycle.

The HPLC profiles of the individual fractions also differ considerably from one another in this example, and individual peptides are enriched visibly in different fractions.

In each embodiment the method according to the invention delivers peptide fractions which are novel in terms of their form, have advantageous functional properties and have a much higher application value than the used raw materials. The separation methods explained above are also able, in their presented combinations and applications, to unlock the great functional potential of protein hydrolysates for broad use.

By varying the process parameters and combining a number of chromatography processes under different conditions, more new peptide mixtures can be produced, up to pure peptides. The method according to the invention and the products resulting therefrom constitute a considerable added value from secondary fractions of food and agricultural production which can otherwise only be valued as inferior, or cannot be valued at all. All sub-fractions having no functional properties sufficient for a specific application can be fed back at least into the original use of the raw materials, and will generally also be valorised by the enzymatic digestion, but are by no means useless.

The elutions of the chromatographic separations are generally all performed with water without further additives; a regeneration of the chromatography columns with chemicals is not necessary. Both the used separation resins for the chromatography processes, known by their use for example in water processing, and the used enzymes are commercially available from the feed and food applications.

1. A method for producing an enriched peptide fraction from a protein-containing raw material, comprising separating protein hydrolysates, in accordance with, their physiochemical properties, by chromatography via stationary phases with an aqueous solution as eluent.

2. The method according to claim 1, wherein before chromatography, the raw materials are set to peptide sizes from 1 to 100 amino acids in a hydrolysis with specific proteases.

3. The method according to claim 1, wherein before chromatography, the method comprises setting a pH value, a concentration and/or a clarification of the protein hydrolysates.

4. The method according to claim 1, wherein the method comprises using as a stationary phase for the chromatography technical adsorbers or ion exchangers without functionalisations or with neutral hydrophilic, adic or basic functionalisations.

5. The method according to claim 1, wherein the method comprises using separation resins as adsorbers as a stationary phase for the chromatography.

6. The method according to claim 5, wherein the separation resins are macroporous or gel-like polymer resins.

7. The method according to claim 1, wherein the method comprises using granulated carbon or pyrolysed separation resins as adsorbers as a stationary phase for the chromatography.

8. The method according to claim 1, wherein the method comprises obtaining a charge solution from the protein-containing raw material which a charge solution has a concentration from 5% to 50% of dry substance.

9. The method according to claim 1, wherein the stationary phases have degrees of cross-linking that have a molecular sieve effect between 300 Da and 10,000 Da.

10. The method according to claim 1, wherein the flow rate of the chromatography is between 0.2 and 4 bed volume per hour.

11. The method according to claim 1, wherein the elution temperature is between −4° C. and 98° C.

12. The method according to claim 10, wherein the elution temperature is between 7020 C. and 95° C.

13. The method according to claim 1, wherein the eluent is pare water.

14. The method according to claim 1, wherein the elutent comprises a diluted aqueous solution of salt, acid, base, or solvent.

15. The method according to claim 1, wherein the method comprises chromatographically enriching the peptides with respect to predefinable functional properties.

16. The method according to claim 1, wherein the method comprises chromatographically purifying the peptides removing undesired secondary constituents.

17. The method according to claim 15, wherein the method comprises further fractionating the resulting peptide mixture via performing further purification steps up to a selected degree of enrichment of one or more peptides.

18. Enriched peptide fractions, produced according to the method of claim 1.

19. A method of making a feedstuff, a foodstuff, a cosmetic product, or a pharmaceutically active ingredient comprising incorporating the peptides obtained according to claim 1 as functional substances in such feedstuff, foodstuff, cosmetic product or pharmaceutically active ingredient, respectively.

20. The method according to claim 16, wherein the method comprises further fractionating the resulting peptide mixture via performing further purification steps up to a selected degree of enrichment of one or more peptides.

21. The method according to claim 17, wherein the further purification steps are chromatographic purification steps.

22. The method according to claim 20, wherein the further purification steps are chromatographic purification steps.
